(19) United States
(12) Patent Application Publication
(10) Pub. No.: US 2024/0331862 A1
(43) Pub. Date: Oct. 3, 2024

(54) SCREENING METHOD AND INDENDITIES OF BIOMARKERS FOR DIFFERENTIAL DIAGNOSIS OF PARKINSONISM AND/OR COGNITIVE IMPAIRMENT

(71) Applicants: National Taiwan University, Taipei (TW); National Taiwan University Hospital, Taipei (TW); Academia Sinica, Taipei (TW); National Tsing Hua University, Hsinchu (TW)

(72) Inventors: Shau-Ping LIN, Taipei (TW); Ruey-Meei WU, Taipei (TW); Frederick Kin Hing Phoa, Taipei (TW); Ming-Che KUO, Taipei (TW); Yi-Tzang TSAI, Taipei (TW); Jing-Wen HUANG, Hsinchu (TW); Yan-Han LIN, Taipei (TW); Hsiang-Hsuan LIN WANG, Taipei (TW); Chia-Lang HSU, Taipei (TW); Ya-Fang HSU, Taipei (TW); Pin-Jui KUNG, Taipei (TW)

(21) Appl. No.: 18/622,407

(22) Filed: Mar. 29, 2024

Related U.S. Application Data
(60) Provisional application No. 63/455,953, filed on Mar. 30, 2023.

(51) Int. Cl. G16H 50/20; C12Q 1/6806; C12Q 1/6883; G01N 33/68; G16H 10/60

(52) U.S. Cl. CPC G16H 50/20; C12Q 1/6806; C12Q 1/6883; G01N 33/6896; G16H 10/60; C12Q 2600/112; C12Q 2600/158; G01N 2333/4706; G01N 2333/521; G01N 2333/575; G01N 2333/7051; G01N 2333/70596; G01N 2333/726; G01N 2333/775; G01N 2333/82; G01N 2333/90203; G01N 2333/91057; G01N 2333/91091; G01N 2333/91215; G01N 2333/96419; G01N 2333/99; G01N 2800/2821; G01N 2800/2835

(57) ABSTRACT

The present invention provides a data analytic scheme for screening biomarkers for differential diagnosis of the status of Parkinson's disease, Parkinson's disease with mild cognitive impairment, Parkinson's disease dementia, Alzheimer's disease, and/or multiple system atrophy, the methodology implementing the same and the results of the screening thereof. Biomedical Oriented Logistic Dantzig Selector (BOLD Selector) was developed to identify candidate microRNAs and extracellular vesicle proteins effective at discerning between any two of the above mentioned disease categories from profiling results. The prediction models are finalized by establishing logistic regression formula for each pair of patient group differentiation.

### FIELD OF TECHNOLOGY

The present invention relates to a method for differential diagnosis of the status of Parkinson's disease, Parkinson's disease with mild cognitive impairment, Parkinson's disease dementia, and/or multiple system atrophy, and in particular to a method for differential diagnosis of the status of Parkinson's disease, Parkinson's disease with mild cognitive impairment, Parkinson's disease dementia, Alzheimer's disease, and/or multiple system atrophy using screened biomarkers, and the analysis systems thereof. However, the present invention is not limited thereto.

### BACKGROUND

Parkinson's disease (PD) is a progressive, age-related, incurable, and debilitating neurodegenerative disease. Parkinson's disease affects about 1-2% of the population over the age of 65, and patients usually present with motor and non-motor symptoms (NMS), wherein the NMS includes cognitive impairment, sensory disturbance and sleep disorders.

Where an individual with Parkinson's disease has a neurocognitive disorder (NCD), the individual can be classified into cohorts such as Parkinson's disease with mild cognitive impairment (PD-MCI) or PD with dementia (PDD), etc. According to clinically statistical data in Taiwan, it is shown that about 40% of patients meet the criteria for PD-MCI, and about 10% of the patients develop PDD in the early stage of the disease; and about 80% of the patients develop PDD in the late stage of the disease.

The diagnostic criteria for PDD and PDD-MCI relies on the administration of extensive neuropsychological tests to PD patients, a process that is time-intensive and requires specialized expertise from psychological professionals. Additionally, clinical practice often employs neuroimaging modalities, such as MRI and FDG-PET, which are resource-demanding and costly.

### SUMMARY

Based on the aforementioned content, the inventor believes that there is currently lacking an effective clinical detection method for early diagnosis of Parkinson's disease complicated with cognitive impairment. Therefore, it is necessary to find a reliable biomarker and provide corresponding drug treatment.

In view of this, a purpose of the present invention is to provide a method for identifying a biomarker for differential diagnosis of Parkinson's Disease (PD), Parkinsonism and/or cognitive impairment, comprising:
a) acquiring plasma samples of a plurality of individuals to obtain a plurality of relevance data of these individuals, and grouping the individuals based on the relevance data;
b) isolating ribonucleic acids containing micro ribonucleic acids (microRNAs) and extracellular vesicular proteins (EV proteins) from the plasma samples of the individuals, and quantitating all microRNAs and up to 4700 extracellular vesicular proteins by small RNA sequencing and LC-MS/MS analysis, respectively;
c) using a Biomedical Oriented Logistic Dantzig Selector (BOLD Selector) to identify at least one candidate microRNA or at least one candidate extracellular vesicle protein from the identification and quantitative profiling result described in b), to differentiate the two chosen patient groups; and
d) calculating a logistic regression formula according to the candidate microRNA(s) and the candidate extracellular vesicle protein(s) to establish a prediction model, and using the prediction model to predict the status of Parkinson's disease, Parkinson's disease with or without cognitive impairment, and/or Parkinson's disease dementia in these individuals.

In some embodiments, in the aforementioned step a), the types of grouping of these individuals comprise: Parkinson's Disease patients with normal cognition ability (no Dementia) (PDND), PD patients with mild cognitive impairment (PD-MCI), Parkinson's Disease Dementia (PDD), Multiple system atrophy (MSA), Alzheimer's disease (AD), and healthy individuals (HC).

In some embodiments, the relevance data is selected from a group consisting of: Movement disorder society-Unified Parkinson's disease rating scale (MDS-UPDRS), Montreal Cognitive Assessment (MoCA) and Mini-mental status examination (MMSE), Unified MSA Rating Scale (UMSARS), physical data and medical history data.

In some embodiments, the physical data comprises age, gender, education level, living habits, diet and exercise habits, and the medical history data comprises medication records, age of onset of Parkinson's disease, and disease duration of Parkinson's disease.

In some embodiments, the microRNA is selected from a group consisting of: miR-203a-3p, miR-626, miR-662, miR-3182, miR-4274, miR-4295, hsa-miR-3173-3p, miR-4306, miR-452-3p, hsa-miR-758-5p, hsa-miR-1197, hsa-miR-208b-5p, hsa-miR-4507, hsa-miR-648, hsa-miR-92b-5p, hsa-miR-3667-3p, hsa-miR-3689a-5p, hsa-miR-3912-3p, hsa-miR-5187-3p, hsa-miR-548b-5p, hsa-miR-519d-5P and hsa-miR-551b-3p.

In some embodiments, the extracellular vesicle protein is selected from a group consisting of: TAOK1 (Serine/threonine-protein kinase TAO1), LCAT (Lecithin cholesterol acyl transferase), CSEIL (Cellular Apoptosis Susceptibility protein, also known as CAS), CRKL (CRK-like proto-oncogene, an adaptor protein), SERPINA4 (Serpin Family A Member 4, also known as Kallistatin), APOE (Apolipoprotein E), ABCC4 (ATP-binding cassette subfamily C member 4), ALDH4A1 (aldehyde dehydrogenase 4 family member A1), TINAGL1 (Tubulointerstitial Nephritis Antigen Like 1), CXCR1 (a chemokine (C-X-C motif) receptor), SWAP70 (Switching B Cell Complex Subunit, 70 kDa), ADGRL2 (Adhesion G Protein-Coupled Receptor L2), Synaptobrevin homolog YKT6, CIDEB (Cell death-inducing DFFA-like effector B), CD96, GLTPD2, CD69, SLC22A23, Tspan15 (transmembrane protein 15), TTC7B, ST3GAL6 (ST3 Beta-Galactoside Alpha-2,3-Sialyltransferase 6), SAMD9, TTC7B, GNB1, ACTBL2 (actin beta like 2), DOK3 (docking protein 3), eIF3B (eukaryotic initiation factor 3), IQGAP1 (IQ domain GTPase-activating protein 1), RPL18A (human 60S ribosomal protein L18a), CLCN5 (Chloride Channel Protein 5), MME (membrane metalloendopeptidase), PUS1, ADIPOQ (Adiponectin), MAP2K6 (Dual Specificity Mitogen-activated Protein Kinase 6), ACTR10, CBLN4 (Cerebellin 4), Epsin 1 (endocytosis accessory protein 1, also known as EPN1), FUCA2 (Alpha-L-fucosidase 2), SNX8, CD3D (CD3 δ subunit of T cell receptor complex), FCGRT, LRRFIP2 (LRR binding FLII interacting protein 2), ARFLP5 (ADP-ribosylation Factor-like Protein 5A), SLC6A4, ARF6 (Switch II GTPase protein) and ATP6V0D1 (ATPase H+ transporting V0 subunit d1).

In some embodiments, before performing the step c), the method further comprises: conducting a data pre-processing step to obtain a processed dataset for the Biomedical Oriented Logistic Dantzig Selector; wherein, when at least one data is missing from the processed dataset, a minimum reading value in other data is inspected and selected in a sample corresponding to the missing data, and an interval between the minimum reading value and zero is uniformly cut to obtain an imputed value, which is then used for filling up the missing data by the overall average of candidates without missing values.

In some embodiments, in the step c), the method further comprises: providing an optimized tuning parameter, and then using the Biomedical Oriented Logistic Dantzig Selector to analyze and identify all factors with non-zero coefficients and the shrink-to-zero position being greater than or equal to the optimized tuning parameter on a delta axis, so as to screen the candidate microRNA from the processed microRNA dataset, and screen the candidate extracellular vesicle protein from the extracellular vesicle protein profile.

In some embodiments, in the step d), the Parkinson's disease and/or Parkinsonism is selected from a group consisting of: Parkinson's Disease patients with normal cognition ability (no Dementia) (PDND), PD patients with mild cognitive impairment (PD-MCI), Parkinson's Disease Dementia (PDD), Multiple system atrophy (MSA), Alzheimer's disease (AD), and healthy individuals (HC).

In some embodiments, in the step d), the logical regression formula adopts a combination of weighted value of a set of microRNAs, or a combination of weighted value of a set of extracellular vesicle proteins.

In some embodiments, after the step d) further comprises: a step of conducting 5-fold iterations of cross-validation on the prediction model.

In some embodiments, the cross-validation step comprises training the prediction model to evaluate the predictive ability of the prediction model for the status of Parkinson's disease, Parkinson's disease with mild impairment and/or Parkinson's disease dementia compared to the grouping results of the individuals in the step a).

In some embodiments, the cross-validation step comprises a detection of the prediction model, wherein the statistical indicators of the detection comprises: sensitivity, specificity, accuracy and area under ROC curve (AUC).

In some embodiments, the method for screening a biomarker for differential diagnosis of the status of Parkinson's Disease (PD), and/or Parkinsonism is implemented by a computation system.

The other purpose of the present invention is to provide a data analytic scheme for executing the aforementioned method, which executes the method of screening a biomarker for differential diagnosis of the status of Parkinson's Disease (PD), Parkinson's disease with or without cognitive impairment and/or Parkinson's disease dementia.

The other purpose of the present invention is to provide biomarkers, which is for differential diagnosis of the status of Parkinson's disease, Parkinson's disease with or without cognitive impairment and/or Parkinson's disease dementia. Wherein, the biomarker is a microRNA and/or an extracellular vesicle protein. In some embodiments, the biomarkers are those screened microRNA as mentioned above. In some embodiments, the biomarkers are those screened extracellular vesicle proteins as mentioned above.

In view of the above, the present invention establishes a method to identify biomarkers from relative comprehensive plasma EV protein and/or microRNA profiling for differential diagnosis of the status of Parkinson's disease, Parkinson's disease with or without cognitive impairment and/or Parkinson's disease dementia, and a data analytic scheme for implementing the method to screen a biomarker related to Parkinson's disease, Parkinsonism and cognitive impairment. A prediction model established by the aforementioned method can be used as a basis for determining whether a biomarker such as a microRNA and an EV protein can effectively distinguish subtypes of Parkinson's disease. Furthermore, the screened biomarker has the potential to be applied in detection technology to fill the medical needs for early diagnosis of patients with Parkinson's disease, and the aforementioned candidate biomarkers can be used for differential diagnosis and grouping of patients with Parkinsonism, so that a right medicine can be prescribed for the patients as early as possible for prevention and treatment.

### BRIEF DESCRIPTION OF THE DRAWINGS

FIG. 1 is a schematic diagram of data results of a preferred embodiment of the present invention, illustrating the results of candidate microRNAs screened by a BOLD selector algorithm under the condition that an optimized tuning parameter is 8.6777.

FIG. 2 is a schematic diagram of data results of a preferred embodiment of the present invention, illustrating the ROC analysis results obtained by a prediction model under 5-fold cross-validation, wherein an average AUC value is shown to be approximately 0.8.

FIG. 3 is a schematic diagram of data results of a preferred embodiment of the present invention, illustrating the average AUC value obtained by the prediction model under 5-fold cross-validation.

FIG. 4 is a schematic diagram of data results of a preferred embodiment of the present invention, illustrating the results of candidate microRNAs screened by a BOLD selector algorithm under the condition that an optimized tuning parameter is 2.7002. The schematic diagram on the left side of FIG. 5 is a schematic diagram of data results of a preferred embodiment of the present invention, which shows that in the screening stage, the expression level of TAOK1 has statistical significance between a group with cognitively normal (HC and PDND) and a group with cognitive impairment (PDD and PD-MCI). The schematic diagram on the right side of FIG. 5 is a schematic diagram of data results of a preferred embodiment of the present invention, which shows that in the screening stage, the expression level of TAOK1 has statistical significance between a group with cognitively normal (HC) and a group with cognitive impairment (AD and MCI).

FIG. 6 is a schematic diagram of data results of a preferred embodiment of the present invention, which shows that in the validation stage, the expression level of TAOK1 has statistical significance between a group with cognitively normal (HC and PDND) and a group with cognitive impairment (PDD and AD).

### DESCRIPTION OF THE EMBODIMENTS

For a more complete and clear disclosure of the utilized technical content, creative purpose and achieved effect of the present disclosure, they are described in detail hereafter, and please refer to the disclosed drawings and reference numbers.

### Terminology

All technical and scientific terms used herein have the same meaning as commonly understood by those of ordinary skills in the art to which the present invention belongs, unless otherwise defined. The following terms used throughout the present application shall have the following meanings.

The terms used in this specification shall be broadly encompassed within the scope of the present invention, and the specific context of each term is the same as its general meaning in the relevant art. In this specification, the specific terms used when describing the present invention will be explained hereafter or elsewhere in this specification, so as to help those of skills in the art to understand the relevant description of the present invention. In the same context, the same term has the same scope and meaning. Furthermore, since there is more than one way to express the same thing, the terms discussed in this specification may be replaced with alternative terms and synonyms, and no special meaning is expressed in this specification regardless of whether a certain term is specified or discussed. Although this specification provides synonyms for some terms, the use of one or more synonyms does not exclude the use of other synonyms.

As used in this specification, “a”, “an” and “the” may be construed as plural, unless the context clearly indicates otherwise. “or” used herein represents “and/or”. As used herein, “comprising or including” means not excluding the presence of or addition of one or more other components, steps, operations, and/or elements to the stated components, steps, operations, and/or elements. The “comprising”, “including”, “containing”, “encompassing” and “having” described herein can also be substituted for each other without limitation. “a” and “an” means that the number of a grammatical object of the term is one or more than one (i.e., at least one).

“Relevance data” used in this specification refers to clinical diagnostic data, physical data and/or medical history data from an individual. Clinical diagnostic items include, but are not limited to: Unified Parkinson's disease rating scale (UPDRS), Movement disorder society-Unified Parkinson's disease rating scale (MDS-UPDRS), Montreal Cognitive Assessment (MoCA), Mini-mental status examination (MMSE), Unified Multiple System Atrophy Rating Scale (UMSARS), and detection of biomarkers in blood. The physical data includes, but is not limited to: age, age at study, gender, education level, living habits, diet, exercise habits, and smoking habits. The medical history data includes, but is not limited to: medication records, levodopa equivalent daily dose (LEDD), age of onset of Parkinson's disease, disease duration of Parkinson's disease, family medical history, and degree of exposure to toxins.

“Movement disorder society-Unified Parkinson's disease rating scale (MDS-UPDRS)” used in this specification refers to a modified version of the UPDRS, which is developed to evaluate multiple aspects of Parkinson's disease, including: motor and non-motor daily life experiences and motor complications.

“Sample” used in this specification refers to fluid or tissue samples from an individual, including but not limited to: saliva, whole blood (blood), serum, plasma, sputum, urine, semen, feces, nasal swabs, tear and tissue sections.

The “microRNA” used in this specification refers to a functional non-coding RNA molecule of about 22 nucleotides in length. It is produced from its precursor RNA by the action of a protein complex including Dicer and Drosha. It can regulate gene expression at a post-transcriptional level by binding to a partial complementary site in a 3′ untranslated region (3′ UTR) of a target gene, thereby inhibiting translation, inducing mRNA degradation, or both. The microRNA plays an important role in many biological processes (including immune responses, cell cycles, cell metabolism and cell death), and it is gradually gaining clinical attention of researchers as a potential biomarker for cancer classification and differential diagnosis of disease status (including neurodegenerative diseases).

“Extracellular vesicles” used in this specification include, but are not limited to, “cytosomes” and “exosomes”.

“Extracellular vesicle protein” used in this specification refers to a protein carried by an extracellular vesicle secreted from cells.

The “processed microRNA dataset” and “extracellular vesicle protein profile” used in this specification refers to a pre-processed dataset comprising identification and quantitative data of microRNAs generated after RNA sequencing, and the profiling data comprising identification and quantification of extracellular vesicle proteins generated after mass spectrometry analysis of a sample, respectively.

The “prediction model” used in this specification is a type of machine learning model, and the “logistic regression formula” used in this specification refers to a maximum likelihood estimation with bias reduction method.

The “prediction model predicts the status of the Parkinson's disease, Parkinson's disease with or without cognitive impairment, and/or Parkinson's disease dementia” of an individual used in this specification means that the prediction model predicts that the individual belongs to which classification group of Parkinson's disease and Parkinsonism and/or predicts the status of cognitive impairment of the individual; wherein the types of grouping include but are not limited to cognitively normal, cognitive impairment, PD, non-PD, and any combination thereof. The aforementioned grouping types include, but are not limited to: Parkinson's Disease patients with normal cognition ability (no Dementia) (PDND), PD patients with mild cognitive impairment (PD-MCI), Parkinson's Disease Dementia (PDD), Multiple system atrophy (MSA), Alzheimer's disease (AD), and healthy individuals (HC).

The “missing data” used in this specification refers to missing values that are less than a threshold and thus not detected, for example those expressed as NA in the detection results.

The “uniformly cut” used in this specification refers to uniformly cutting into equal parts. Specifically, in a sample corresponding to the missing data, a minimum reading value in other data is inspected and selected, and an interval between the minimum reading value and zero is uniformly cut to obtain an imputed value.

Method for Screening a Biomarker for Differential Diagnosis of Parkinson's Disease and/or a Status of Parkinsonism, and Data Analytic Scheme Thereof

According to some embodiments, the present invention provides method for screening a biomarker for differential diagnosis of the status of Parkinson's disease, Parkinsonism, and a cognitive impairment, which includes:
a) acquiring plasma samples of a plurality of individuals to obtain a plurality of relevance data of these individuals, and grouping the individuals based on the relevance data;
b) isolating ribonucleic acids containing micro ribonucleic acids (microRNAs) and extracellular vesicular proteins from the plasma samples of the individuals, and analyzing and identifying to obtain a microRNA dataset and extracellular vesicular protein profiling data;
c) using a Biomedical Oriented Logistic Dantzig Selector (BOLD Selector) to screen at least one candidate microRNA from the microRNA dataset, and to screen at least one candidate extracellular vesicle protein from the extracellular vesicle protein profile; and
d) calculating a logistic regression formula according to the candidate microRNA and the candidate extracellular vesicle protein to establish a prediction model, and using the prediction model to predict the status of Parkinson's disease, Parkinsonism, and cognitive impairment in these individuals.

In some embodiments, in the aforementioned step a), the type of grouping of these individuals can be arbitrarily selected according to the following different cohort types, wherein the type of grouping of these individuals includes, but is not limited to:
i) cognitively normal and cognitive impairment, wherein the cognitively normal includes: healthy individuals (HC) and/or Parkinson's Disease patients with normal cognition ability (PDND), and wherein the cognitive impairment includes PD patients with mild cognitive impairment (PD-MCI), Parkinson's Disease Dementia (PDD), Multiple system atrophy (MSA) and Alzheimer's disease (AD); and
ii) PD and/or non-PD, wherein PD can be further divided into Parkinson's Disease patients with normal cognition ability (PDND) and non PDND, wherein the non PDND is further divided into PD patients with mild cognitive impairment (PD-MCI) and Parkinson's Disease Dementia (PDD). Besides, non-PD can be divided into healthy individuals (HC) and Multiple system atrophy (MSA).

According to some embodiments, in the aforementioned step a), the type of grouping of these individuals includes: Parkinson's Disease patients with normal cognition ability (PDND), PD patients with mild cognitive impairment (PD-MCI), Parkinson's Disease Dementia (PDD), Multiple system atrophy (MSA), Alzheimer's disease (AD), and healthy individuals (HC).

According to some embodiments, the relevance data is selected from a group consisting of: Movement disorder society-Unified Parkinson's disease rating scale (MDS-UPDRS), Montreal Cognitive Assessment (MoCA), Mini-mental status examination (MMSE), physical data, and medical history data.

According to some embodiments, the Montreal Cognitive Assessment (MoCA) is used for quickly determining the cognitive performance of the individuals, wherein the total score after evaluation is used for grouping the subjects. A cognitive domain includes: visuospatial, naming, attention, language, abstraction, memory and orientation domains. According to some embodiments, HC subjects and PDND patients should meet a total MoCA score equal to or higher than 26. PD-MCI patients should meet a total MoCA score falling within the range of 22 to 25. PDD patients should meet a total MoCA score equal to or lower than 21.

According to some embodiments, the physical data includes age, age at study, gender, education level, living habits, diet and exercise habits, and the medical history data includes medication records, age of onset and duration of illness.

According to some embodiments, the microRNA is selected from a group consisting of: miR-203a-3p, miR-626, miR-662, miR-3182, miR-4274, miR-4295, hsa-miR-3173-3p, miR-4306, miR-452-3p, hsa-miR-758-5p, hsa-miR-1197, hsa-miR-208b-5p, hsa-miR-4507, hsa-miR-648, hsa-miR-92b-5p, hsa-miR-3667-3p, hsa-miR-3689a-5p, hsa-miR-3912-3p, hsa-miR-5187-3p, hsa-miR-548b-5p, hsa-miR-519d-5P and hsa-miR-551b-3p. Table 1 below shows base sequences from the 5′ terminus to the 3′ terminus of the aforementioned RNA biomarkers, and deposit numbers thereof.

TABLE 1
RNA biomarkers

| | | miRBase | |
|---|---|---|---|
| | | Deposit | |
| Group | RNA biomarkers | Number | Sequence |
| PD-MCI | miR-203a-3p | MIMAT0000264 | gugaaauguuuaggaccacuag |
| vs. PDND | (hsa-miR-203a-3p) | | |
| | miR-16-5p | MIMAT0000069 | uagcagcacguaaauauuggcg |
| | (hsa-miR-16-5p) | | |
| | miR-626 | MIMAT0003295 | agcugucugaaaaugucuu |
| | (hsa-mir-626) | | |
| | miR-662 | MIMAT0003325 | ucccacguuguggcccagcag |
| | (hsa-miR-662) | | |
| | miR-3182 | MIMAT0015062 | gcuucuguaguguaguc |
| | miR-4274 | MIMAT0016906 | cagcagucccucccccug |
| | miR-4295 | MIMAT0016844 | cagugcaauguuuuccuu |
| MSA vs. | hsa-miR-3173-3p | MIMAT0015048 | aaaggaggaaauaggcaggcca |
| HC (TMM) | hsa-miR-4292 | MIMAT0016919 | ccccugggccggccuugg |
| | hsa-miR-140-3p | MIMAT0004597 | uaccacaggguagaaccacgg |
| | hsa-miR-16-2-3p | MIMAT0004518 | ccaauauuacugugcugcuuua |
| | hsa-miR-3937 | MIMAT0018352 | acaggcggcuguagcaauggggg |
| | hsa-miR-5093 | MIMAT0021085 | aggaaaugaggcuggcuaggagc |
| MSA vs. | miR-4306 | MIMAT0016858 | uggagagaaaggcagua |
| PDND | (hsa-miR-4306) | | |
| (TMM) | miR-452-3p | MIMAT0001636 | cucaucugcaaagaaguaagug |
| | (hsa-miR-452-3p) | | |
| PDND vs. | hsa-miR-758-5p | MIMAT0022929 | gaugguugaccagagagcacac |
| HC | hsa-miR-1197 | MIMAT0005955 | uaggacacauggucuacuucu |
| (ANOVA) | | | |
| MSA vs. | hsa-miR-208b-5p | MIMAT0026722 | aagcuuuuugcucgaauuaugu |
| HC (RPM) | hsa-miR-4507 | MIMAT0019044 | cuggguugggcugggcuggg |
| | hsa-miR-3173-3p | MIMAT0015048 | aaaggaggaaauaggcaggcca |
| | hsa-miR-556-5p | MIMAT0003220 | gaugagcucauuguaauaugag |
| | hsa-miR-5093 | MIMAT0021085 | aggaaaugaggcuggcuaggagc |
| MSA vs. | hsa-miR-648 | MIMAT0003318 | aagugugcagggcacuggu |
| PDND | hsa-miR-92b-5p | MIMAT0004792 | agggacgggacgcggugcagug |
| (RPM) | hsa-miR-4306 | MIMAT0016858 | uggagagaaaggcagua |
| | hsa-miR-452-3p | MIMAT0001636 | cucaucugcaaagaaguaagug |
| | hsa-miR-3653-5p | MIMAT0032110 | ccuccugaugauucuucuuc |
| | hsa-miR-4782-3p | MIMAT0019945 | ugauugucuucauaucuagaac |
| | hsa-miR-302d-5p | MIMAT0004685 | acuuuaacauggaggcacuugc |
| | hsa-miR-379-3p | MIMAT0004690 | uauguaacaugguccacuaacu |
| | hsa-miR-412-3p | MIMAT0002170 | acuucaccugguccacuagccgu |
| | hsa-miR-4296 | MIMAT0016845 | augugggcucaggcuca |
| | hsa-miR-6747-3p | MIMAT0027395 | uccugccuuccucugcaccag |
| PD vs. | hsa-miR-3667-3p | MIMAT0018090 | accuuccucuccaugggucuuu |
| MSA + HC | hsa-miR-3689a-5p | MIMAT0018117 | ugugauaucaugguuccuggga |
| (PRM) | hsa-miR-3912-3p | MIMAT0018186 | uaacgcauaauauggacaugu |
| | hsa-miR-5187-3p | MIMAT0021118 | acugaauccucuuuuccucag |
| | hsa-miR-548b-5p | MIMAT0004798 | aaaaguaauugugguuuuggcc |
| PD vs. HC | hsa-miR-519d-5p | MIMAT0026610 | ccuccaaagggaagcgcuuucuguu |
| (RPM) | hsa-miR-551b-3p | MIMAT0003233 | gcgacccauacuugguuucag |

According to some embodiments, the extracellular vesicle protein is selected from a group consisting of: TAOK1 (Serine/threonine-protein kinase TAO1), LCAT (Lecithin cholesterol acyl transferase), CSEIL (Cellular Apoptosis Susceptibility protein, also known as CAS), CRKL (CRK-like proto-oncogene, adaptor protein), SERPINA4 (Serpin Family A Member 4, also known as Kallistatin), APOE (Apolipoprotein E), ABCC4 (ATP-binding cassette subfamily C member 4), ALDH4A1 (aldehyde dehydrogenase 4 family member A1), TINAGL1 (Tubulointerstitial Nephritis Antigen Like 1), CXCR1 (chemokine (C-X-C motif) receptor), SWAP70 (Switching B Cell Complex Subunit, 70 kDa), ADGRL2 (Adhesion G Protein-Coupled Receptor L2), Synaptobrevin homolog YKT6, CIDEB (Cell death-inducing DFFA-like effector B), CD96, GLTPD2 (glycolipid transfer protein domain containing 2), CD69, SLC22A23 (solute carrier family 22 member 23), Tspan15 (transmembrane protein 15), TTC7B (tetratricopeptide repeat domain 7B), ST3GAL6 (ST3 Beta-Galactoside Alpha-2,3-Sialyltransferase 6), SAMD9 (sterile alpha motif domain containing 9), GNB1 (G protein subunit beta 1), ACTBL2 (actin beta like 2), DOK3 (docking protein 3), eIF3B (eukaryotic initiation factor 3), IQGAP1 (IQ domain GTPase-activating protein 1), RPL18A (human 60S ribosomal protein L18a), CLCN5 (Chloride Channel Protein 5), MME (membrane metalloendopeptidase, PUS1 (pseudouridine synthase 1), ADIPOQ (Adiponectin), MAP2K6 (Dual Specificity Mitogen-activated Protein Kinase 6), ACTR10 (actin related protein 10), CBLN4 (Cerebellin 4), Epsin 1 (endocytosis accessory protein 1, also known as EPN1), FUCA2 (alpha-L-fucosidase 2), SNX8 (sorting nexin 8), CD3D (CD3 δ subunit of T cell receptor complex), FCGRT (Fc gamma receptor and transporter), LRRFIP2 (LRR binding FLII interacting protein 2), ARFLP5 (ADP-ribosylation Factor-like Protein 5A), SLC6A4, ARF6 (ADP ribosylation factor 6, also known as Switch II GTPase protein), ATP6V0D1 (ATPase H⁺ transporting V0 subunit d1), LAMB4 (pseudouridine synthase 1Laminin subunit β4), PGLYRP1 (peptidoglycan recognition protein 1), KCTD12 (potassium channel tetramerization domain containing 12), NIPSNAP1 (nipsnap homolog 1), SDR9C7 (Short-chain dehydrogenase/reductase family 9C member 7), ANTXR2 (Anthrax toxin receptor 2), VAT1 (Synaptic vesicle membrane protein VAT-1 homolog), TBC1D1 (TBC1 domain family member 1), PRPS1 (Ribose-phosphate pyrophosphokinase 1), SERPINA6 (Serpin family A member 6), ITGA11 (Integrin alpha-11), SMIM5 (Small integral membrane protein 5), TOR3A (Torsin-3A), PDGFC (Platelet-derived growth factor C) and SIGIRR (Single Ig IL-1-related receptor). Table 2 below lists the amino acid sequences of the aforementioned protein biomarkers and deposit numbers thereof.

TABLE 2
Protein biomarkers

| | | UniProt |
|---|---|---|
| Group | Protein biomarkers | accession number |
| MSA vs. HC | Lecithin-cholesterol acyltransferase | P04180 |
| | (LCAT) | |
| MSA vs. HC | Serpin family A member 4 (SERPINA4) | P29622 |
| MSA vs. HC | Cellular apoptosis susceptibility protein | P55060 |
| | (chromosome segragation 1-like, CSEIL) | |
| MSA vs. HC | Adapter protein (CRKL) | P46109 |
| MSA vs. PD | Serpin family A member 4 (SERPINA4) | P29622 |
| MSA vs. PD | Apolipoprotein E (ApoE) | P02649 |
| MSA vs. PD | ATP-binding cassette subfamily C | O15439 |
| | member 4 (ABCC4) | |
| MSA vs. PD | Aldehyde dehydrogenase 4 family | P30038 |
| | member A1 (ALDH4A1) | |
| PD vs. HC | Tubulointerstitial nephritis antigen like 1 | Q9GZM7 |
| | (TINAGLI) | |
| PD vs. HC | Chemokine (C-X-C motif) receptor | P25024 |
| | (CXCR1) | |
| PD vs. HC | Switching B cell complex subunit | Q9UH65 |
| | SWAP70 (SWAP70) | |
| PD vs. HC | Adhesion G protein-coupled receptor L2 | O95490 |
| | (ADGRL2) | |
| PD vs. HC | Dual Specificity Mitogen-activated | P52564 |
| | Protein Kinase 6 (MAP2K6) | |
| PD vs. HC | Laminin subunit ß4 (LAMB4) | A4DOS4 |
| PD vs. HC | Peptidoglycan recognition protein 1 | O75594 |
| | (PGLYRP1) | |
| PD vs. HC | Membrane metalloendopeptidase (MME) | P08473 |
| PD vs. HC | Potassium channel tetramerisation domain | Q96CX2 |
| | containing protein 12 (KCTD12) | |
| PD vs. HC | NIPSNAP1 | Q9BPW8 |
| PD vs. HC | Short-chain dehydrogenase/reductase | Q8NEX9 |
| | family 9C member 7 (SDR9C7) | |
| PD vs. HC | ANTXR cell adhesion molecule 2 | P58335 |
| | (ANTXR2) | |
| PD vs. HC | Vesicle amine transporter 1 (VAT1) | Q99536 |
| PD vs. HC | TBC1 domain family member 1 | Q86TI0 |
| | (TBC1D1) | |
| PDND vs. | Synaptobrevin homolog (Ykt6) | O15498 |
| PD-MCI + PDD | | |
| PDND vs. | Cell-death-inducing DFFA-like effector B | Q9UHD4 |
| PD-MCI + PDD | (CIDEB) | |
| PDND vs. | Phosphoribosyl pyrophosphate synthetase | P60891 |
| PD-MCI + PDD | 1 (PRPS1) | |
| PDND vs. | CD96 | P40200 |
| PD-MCI + PDD | | |
| PDND vs. | Serpin family A member 6 (SERPINA6) | P08185 |
| PD-MCI + PDD | | |
| PDND vs. | Integrin subunit all (ITGA11) | Q9UKX5 |
| PD-MCI + PDD | | |
| PDND vs. | Small integral membrane protein 5 | Q71RC9 |
| PD-MCI + PDD | (SMIM5) | |
| PDND vs. | Torsin family 3 member A (TOR3A) | Q9H497 |
| PD-MCI + PDD | | |
| PD-MCI vs. | Cell-death-inducing DFFA-like effector B | Q9UHD4 |
| PDND | (CIDEB) | |
| PD-MCI vs. | CD96 | P40200 |
| PDND | | |
| PD-MCI vs. | Synaptobrevin homolog (Ykt6) | 015498 |
| PDND | | |
| PD-MCI vs. | Glycolipid transfer protein domain | A6NH11 |
| PDND | containing 2 (GLTPD2) | |
| PD-MCI vs. | Platelet-derived growth factor C (PDGFC) | Q9NRA1 |
| PDND | | |
| PD-MCI vs. | Single Ig and TIR domain containing | Q6IA17 |
| PDND | (SIGIRR) | |
| PD-MCI vs. | Phosphoribosyl pyrophosphate synthetase | P60891 |
| PDND | 1 (PRPS1) | |
| MCI vs. HC | CD69 | Q07108 |
| MCI vs. HC | Solute carrier family 22 member 23 | A1A5C7 |
| | (SLC22A23) | |
| MCI vs. HC | Transmembrane protein 15 (Tspan15) | O95858 |
| MCI vs. HC | TTC7B | Q86TV6 |
| MCI vs. HC | ST3β-Galactoside α-2,3-Sialyltransferase | Q9Y274 |
| | 6 (ST3GAL6) | |
| AD + MCI vs. | SAMD9 | Q5K651 |
| HC | | |
| AD + MCI vs. | TTC7B | Q86TV6 |
| HC | | |
| AD + MCI vs. | GNB1 | P62873 |
| HC | | |
| AD + MCI vs. | Actin beta like 2 (ACTBL2) | Q562R1 |
| HC | | |
| AD + MCI vs. | Docking Protein 3 (DOK3) | Q7L591 |
| HC | | |
| PD vs. | Eukaryotic translation initiation factor 3 | P55884 |
| HC + MSA | (eIF3B) | |
| PD vs. | SLC6A4 | P31645 |
| HC + MSA | | |
| PD vs. | IQ motif containing GTPase-activating | P46940 |
| HC + MSA | protein 1 (IQGAP1) | |
| PD vs. | Tubulointerstitial nephritis antigen like 1 | Q9GZM7 |
| HC + MSA | (TINAGLI) | |
| PD vs. | Human 60S ribosomal protein L18a | Q02543 |
| HC + MSA | (RPL18A) | |
| PD vs. | ATP-binding cassette subfamily C | O15439 |
| HC + MSA | member 4 (ABCC4) | |
| PD vs. | Chloride voltage-gated channel 5 | P51795 |
| HC + MSA | (CLCN5) | |
| PD vs. | Membrane metalloendopeptidase (MME) | P08473 |
| HC + MSA | | |
| PD vs. | PUS1 | Q9Y606 |
| HC + MSA | | |
| PD vs. | Adiponectin (ADIPOQ) | Q15848 |
| HC + MSA | | |
| PD vs. | Dual Specificity Mitogen-activated | P52564 |
| HC + MSA | Protein Kinase 6 (MAP2K6) | |
| PD vs. | ACTR10 | Q9NZ32 |
| HC + MSA | | |
| PD vs. | Cerebellin 4 precursor (CBLN4) | Q9NTU7 |
| HC + MSA | | |
| PD vs. | Endocytic accessory protein 1 (EPN1) | Q9Y613 |
| HC + MSA | | |
| PD vs. | Lecithin-cholesterol acyltransferase | P04180 |
| HC + MSA | (LCAT) | |
| PD vs. | α-L-fucosidase 2 (FUCA2) | Q9BTY2 |
| HC + MSA | | |
| PD vs. | SNX8 | Q9Y5X2 |
| HC + MSA | | |
| PD vs. | CD3 δ subunit (CD3D) of T cell receptor | P04234 |
| HC + MSA | complex | |
| PD vs. non PD | Eukaryotic translation initiation factor 3 | P55884 |
| | (eIF3B) | |
| PD vs. non PD | Tubulointerstitial nephritis antigen like 1 | Q9GZM7 |
| | (TINAGLI) | |
| PD vs. non PD | Adiponectin (ADIPOQ) | Q15848 |
| PD vs. non PD | Fc γ receptor and transporter (FCGRT) | P55899 |
| PD vs. non PD | α-L-fucosidase 2 (FUCA2) | Q9BTY2 |
| PD vs. non PD | ACTR10 | Q9NZ32 |
| AD + MCI vs. | LRR-binding FLII interacting protein 2 | Q9Y608 |
| PD-MCI + PDD | (LRRFIP2) | |
| AD + MCI vs. | ADP-ribosylation factor-like GTPase 5A | Q9Y689 |
| PD-MCI + PDD | (ARL5A) | |
| AD + MCI vs. | LRR-binding FLII interacting protein 2 | Q9Y608 |
| PDND | (LRRFIP2) | |
| AD + MCI vs. | Tubulointerstitial nephritis antigen like 1 | Q9GZM7 |
| PDND | (TINAGLI) | |
| MSA vs. PDND | Adapter protein (CRKL) | P46109 |
| MSA vs. PDND | SLC6A4 | P31645 |
| MSA vs. PDND | ADP-ribosylation factor 6 (ARF6) | P62330 |
| MSA vs. PDND | GNB1 | P62873 |
| MSA vs. PDND | ATP6V0D1 | P61421 |

According to some embodiments, before performing the aforementioned step c), it further includes: conducting a data pre-processing step to obtain a processed dataset for the Biomedical Oriented Logistic Dantzig Selector, wherein, when at least one data is missing from the processed dataset, a minimum reading value in other data is inspected and selected in a sample corresponding to the missing data, and an interval between the minimum reading value and zero is uniformly cut to obtain an imputed value, which is then used for filling up the missing data according to the overall averages of candidates without missing value.

According to some embodiments, in the step c), it further includes: providing an optimized tuning parameter, and then using the Biomedical Oriented Logistic Dantzig Selector to analyze and identify all factors with non-zero coefficients and the shrink-to-zero position being greater than or equal to the optimized tuning parameter on a delta axis, so as to screen the candidate microRNA from the processed microRNA dataset, and screen the candidate extracellular vesicle protein from the extracellular vesicle protein profile.

According to some embodiments, in the aforementioned step d), the Parkinson's disease and/or Parkinsonism is selected from a group consisting of: Parkinson's Disease patients with normal cognition ability, PD patients with mild cognitive impairment, Parkinson's Disease Dementia, and Multiple system atrophy.

According to some embodiments, in the aforementioned step d), the logistic regression formula adopts a combination of weighted value of a set of microRNAs, or a combination of weighted value of a set of extracellular vesicle proteins.

According to some embodiments, after the aforementioned step d), it further includes: a step of conducting at least 5-fold cross-validation on the prediction model. The cross-validation step includes training the prediction model to evaluate the predictive ability of the prediction model for the status of Parkinson's disease and/or Parkinsonism compared to the grouping results of the individuals in step a). In a preferred embodiment, the prediction model undergoes 5-fold cross-validation step.

According to some embodiments, the cross-validation step further includes a detection of the prediction model, wherein the statistical indicators of the detection includes: sensitivity, specificity, accuracy, and area under ROC curve (AUC).

According to some embodiments, the aforementioned method for screening a biomarker for differential diagnosis of the status of Parkinson's disease and/or Parkinsonism is implemented by a computer.

According to some embodiments, the present invention provides a computer system for performing the method for screening a biomarker for differential diagnosis of the status of Parkinson's disease and/or Parkinsonism.

In some embodiments, the individual refers to human being.

In some embodiments, the sample refers to plasma.

### Biomedical Oriented Logistic Dantzig Selector (BOLD)

In some embodiments, a analyzing method of the Biomedical Oriented Logistic Dantzig Selector includes:
a) standardizing the data so that the y-axis has a mean value of 0 and the standard deviation of each column in the factor profiling data is the same;
b) setting an appropriate tuning parameters 8, and solving a linear programming to uniformly cut between 0 and 8 to obtain a corresponding coefficient ß of each factor; and
c) depicting an analysis broken line graph according to the tuning parameters and coefficient of each factor to visualize the results of the BOLD selector, selecting an optimized 8 through 5-fold cross-validation, and using the Biomedical Oriented Logistic Dantzig Selector to analyze and identify all factors with non-zero coefficients and the shrink-to-zero position being greater than or equal to the optimized tuning parameter on the delta axis, so as to screen an important candidate factor.

In some embodiments, screening a candidate biomarker mainly includes the following three steps:
a) pre-processing of missing data
a simple imputation step for handling missing entries in an impute dataset:
There are two possible reasons why there are missing values in the data set. One is that the signal of the sample is lower than the threshold value and cannot be detected by an instrument, and the other is that some specific factor values are all missing. For the latter one (some specific factor values are all missing), they will be excluded from the analysis data of the present application. Furthermore, in the sample corresponding to the missing data, a minimum reading value in other data is inspected and selected, and the interval between the minimum reading value and zero is uniformly cut to obtain an imputed value, which is then used for filling up the missing data. An overall relative mean is used for determining whether the imputed value is large or small. The data set obtained after the aforementioned processing will be applied to the BOLD selector algorithm.
b) quickly screening of an important biomarker from all listed biomarkers:
For selection of the tuning parameters, first the data is substituted into the prediction model and undergoes 5-fold cross-validation to obtain the AUCs value under iterative analysis. The fitness of the prediction model in the 5-fold cross-validation is evaluated by AUC analysis, so as to facilitate the selection of the optimized tuning parameter/optimal tuning parameter with the highest average AUC.
After an optimized tuning parameter is selected, the BOLD selector algorithm is used for analyzing and identifying all factors with non-zero coefficients and the shrink-to-zero position being greater than or equal to the optimized tuning parameter on the delta axis to screen a candidate biomarker from the processed microRNA dataset or extracellular vesicle protein profile.
c) establishment of a logistic regression formula to retain a final candidate biomarker: significant factors (e.g., candidate biomarkers) are ranked and identified, and then the candidate biomarkers are used for calculating a final logistic regression formula.

In some embodiments, the “candidate microRNA” and the “candidate extracellular vesicle protein” are associated with the cognition ability of the individual.

In some embodiments, the expression level of the target miRNA is relative to the level of a reference. The reference is an endogenous reference miRNA, e.g.: miR-16-5p, which has rich intracellular and intercellular contents and is relatively constant in biofluids of different ages.

In some embodiments, the expression level of the miRNA is expressed in terms of a level normalized by a trimmed mean of M-values (TMM).

In some embodiments, the expression level of the miRNA is expressed in terms of a level normalized by reads per million mapped reads (RPM).

In some embodiments, the expression level of the miRNA is expressed in terms of a level normalized by analysis of variance (ANOVA).

In some embodiments, the expression level of miR-203a-3p refers to the level of miR-203a-3p normalized by miR-16-5p.

In some embodiments, the prediction model can be a machine learning model using any algorithm, including but not limited to: logistic regression, a support vector machine, a decision tree, deep neural networks, recurrent neural networks, convolutional neural networks, naive Bayes and random forest.

### EXAMPLES

Hereinafter, the contents disclosed in the present invention will be described with reference to Examples and drawings. However, the disclosure of the present invention is not limited to these embodiments and drawings.

### Example 1. Recruitment of Participants

All patients with Parkinson's disease met the inclusion criteria set out by the UK Parkinson's Disease Society Brain Bank Criteria. Between January 2018 and December 2019, a total of 160 participants were recruited; wherein 58 participants served as the Discovery Cohort (also known as Cohort 1), and the remaining 92 participants served as a Validation Cohort (also known as Cohort 2).

Wherein, in the Discovery Cohort, 17 participants were HC individuals, 10 participants were MSA patients, and 41 participants were PD patients, for a total of 58 participants. These 58 participants were the analyzed subjects for sample isolation and purification to obtain the microRNA dataset and extracellular vesicle protein profiling data.

In the Validation Cohort, 16 participants were HC individuals, 38 participants were MSA patients, and 38 participants were PD patients, for a total of 92 participants. These 92 participants were applied in the step of validating plasma-derived candidate microRNAs and plasma-derived candidate extracellular vesicle proteins.

The aforementioned participants were diagnosed and grouped by the National Taiwan University Hospital (NTUH).

### Example 2: Obtaining a Plurality of Relevance Data from Individuals

The collected data were as follows
1. physical data: including gender and age at study collected from a plurality of individuals;
2. clinical history data: including age of onset and disease duration; and
3. clinical diagnostic items: including Part II of the Unified Multiple System Atrophy Rating Scale (UMSARS), Part III of the Unified Parkinson's disease rating scale (UPDRS), and the Mini-mental status examination (MMSE). Table 3 below listed the relevance data of each cohort.

TABLE 3

| | Cohort 1 | | | Cohort 2 | | |
|---|---|---|---|---|---|---|
| Variables | HCs | MSA | PD | HCs | MSA | PD |
| Number of | 17 | 10 | 41 | 16 | 38 | 38 |
| individuals (n) | | | | | | |
| Age | 72.6 ± 4.4 | 66.6 ± 7.4 | 72.7 ± 6.9 | 69.4 ± 3.3 | 67.0 ± 7.0 | 55.4 ± 14.8 |
| at study | | | | | | |
| Male (n) | 7 (38.9%) | 7 (30.0%) | 23 (56.1%) | 3 (18.8%) | 25 (65.8%) | 22 (57.9%) |
| Age | — | 62.9 ± 7.6 | 65.4 ± 6.4 | — | 61.5 ± 7.1 | 44.5 ± 13.1 |
| of onset | | | | | | |
| disease | — | 4.7 ± 0.8 | 8.3 ± 3.3 | — | 6.5 ± 3.8 | 11.9 ± 7.4 |
| duration | | | | | | |
| Part II of | — | 13.5 ± 12.0 | | — | 14.5 ± 5.8 | — |
| UMSARS | | | | | | |
| Part III of | — | 26.6 ± 14.0 | 20.7 ± 13.2 | — | 33.4 ± 13.9 | 24.3 ± 14.6 |
| UPDRS | | | | | | |
| MMSE | — | 27.3 ± 2.3 | 24.8 ± 3.9 | — | 25.1 ± 2.7 | 29.0 ± 0.0 |

The data for continuous variables was presented as mean ± standard deviation (SD), and the data for categorical variables was presented as frequency (%).

### Example 3: Plasma Collection

10 mL of blood was collected from each individual into a vacuum blood collection tube (BD Vacutainer K2E (EDTA) Plus; Becton Dickinson, USA). The blood was centrifuged at a rotation speed of 2,200×g (swinging bucket, KUBOTA 4000, Japan) at room temperature for 15 min, and a plasma layer was collected within 3 hours.

### Example 4: Sequencing of Plasma RNA

MicroRNAs (less than 200 nucleotides) were isolated from 200-400 μL of the human plasma sample by using a Qiagen miRNeasy Mini reagent kit (Qiagen, Cat. #217004). Plasma miRNA profiling was conducted by constructing a small RNA library with QIAseq miRNA Library Kit and using next-generation sequencing (NGS), wherein single-end microRNA sequencing was conducted on an Illumina NextSeq (Qiagen, #331502) to establish microRNA profiling data. The microRNAs identified above were statistically analyzed to generate a processed microRNA dataset.

### Example 5: Profiling of Extracellular Vesicle Proteins

Plasma was isolated from blood derived from an individual, and subjected to size exclusion-based gravity-flow chromatography by EVSecond L70 column (GL Sciences, Tokyo, Japan) to isolate extracellular vesicles (EVs). Anti-CD9/anti-CD63 or anti-CD9/anti-CD9 sandwich enzyme-linked immunosorbent assay (ELISA) was routinely performed to confirm EV enrichment. Plasma EVs were lysed, followed by Trypsin digestion of the EV-associated proteins.

The resulting peptide was subjected to mass spectrometry analysis of the sample by liquid chromatography-tandem mass spectrometry (LC-MS/MS), e.g., Orbitrap Fusion Lumos or Orbitrap Fusion Lumos combined with a FAIMS device. The MS/MS spectra were queried in the Homo sapiens protein sequence database from SwissProt using Proteome Discoverer 3.0 software (Thermo Scientific), with peptide identification filters set to a “false discovery rate of less than 1%”. A proteomic profile of EVs isolated from an individual's blood plasma was generated, comprising both protein identification and quantification data.

### Example 6. Screening of Candidate Biomarkers (for microRNAs and Extracellular Vesicle Proteins) and Construction of Prediction Models

Before the BOLD Selector algorithm was used for screening candidate microRNAs and extracellular vesicle proteins, numerical inspection in the dataset (e.g.: sequencing and identification results of proteins and microRNAs collected from patients) was conducted.

Table 4 below showed the numerical pre-processing of missing data. According to Table 4, for patient No. 1, there were two pieces of missing data in the protein sequencing and identification results, which were the column of protein 4 and the column of protein 5, respectively. The minimum value in the data of the sample was 20, and the interval from the minimum value 20 to 0 was uniformly cut, so that 0 (as the imputed value) was imputed in the column of protein 4, and 10 was imputed in the column of protein 5, because the averages without missing values of protein 4 and 5 are 40 and 50, respectively, indicating that the missing value of protein 5 should be imputed by a larger value than that of protein 4.

TABLE 4
Pre-processing of missing data

| | 1 | 2 | 3 | 4 | 5 |
|---|---|---|---|---|---|
| 1 | 30 | 50 | 20 | NA | NA |
| | | | | (it was imputed | it was imputed |
| | | | | to 0) | to 10) |
| 2 | 20 | 30 | NA | 40 | NA |
| 3 | 30 | NA | NA | 40 | 50 |
| 4 | NA | 40 | 30 | NA | 50 |

The values in Table 4 were illustrative and were only used for illustrating how to calculate the imputed values to fill up the missing data according to the overall averages of candidates without missing values.

After the aforementioned dataset was subjected to pre-processing of missing data, the processed dataset was used for the subsequent BOLD selector algorithm to screen candidate microRNAs and extracellular vesicle proteins.

The BOLD selector algorithm was used for screening out a plurality of candidate microRNAs from the processed microRNA dataset, and for screening out a plurality of candidate extracellular vesicle proteins from the extracellular vesicle protein profile. An initial logistic regression formula was calculated according to the plurality of candidate microRNAs and candidate extracellular vesicle proteins to establish a prediction model.

After the prediction model was established, the data from Cohort 2 was substituted into the prediction model for model fit-in validation.

Please refer to Table 5 together. Before the cohort dataset of Cohort 2 was substituted into the prediction model, Cohort 2 was first subjected to clinical diagnosis, plasma collection, plasma RNA sequencing and profiling, and profiling of plasma EV proteomes as described above, so as to obtain the cohort data of Cohort 2. The data of Cohort 2 included: clinical diagnosis results, and a processed dataset or profiles generated after sequencing, identification and statistical analysis. The data of Cohort 2 was subjected to 5-fold cross-validation on the prediction model to obtain the AUCs. The fitness of the prediction model in the 5-fold iterations was evaluated by obtaining the average area of AUC, and the optimized tuning parameter (delta value) with the highest average AUC value was selected, as shown in Table 3. After the aforementioned optimized tuning parameter was obtained, then the BOLD selector was used for analyzing and identifying all factors with non-zero coefficients and the shrink-to-zero position being greater than or equal to the optimized tuning parameter on the delta axis to screen candidate biomarkers from the processed dataset or profile. Please refer to Table 5. For example, the BOLD selector ranked the screened biomarkers. For example, the biomarker hsa-miR-3173-3p in Table 5 was screened from the processed microRNA dataset by the BOLD selector and ranked first in a candidate list. Therefore, hsa-miR-3173-3p was used as a biomarker for distinguishing MSA cohorts from HC cohorts. The biomarker SERPINA4 was screened from the extracellular vesicle protein profile by the BOLD selector and ranked No. 1 in the candidate list. Therefore, SERPINA4 was used as a biomarker for distinguishing the MSA cohorts from the PD cohorts.

TABLE 5

| | | | |
|---|---|---|---|
| | | Discovery phase/ | |
| | | Screening phase | |
| | | Comparing the | |
| | | statistical significance | Validation phase |
| | | of biomarker | Comparing the |
| | | expression between | statistical significance |
| | | two cohorts | of biomarker |
| | | (p value) or | expression between |
| | | Ranking of grouping | two cohorts |
| Screened biomarkers | Distinguished cohorts | ability | (p value ) |

Grouping by microRNA

| | | | |
|---|---|---|---|
| miR-203a-3p, | PD-MCI and PDND | | |
| miR-626, miR-662, | | | |
| miR-3182, miR-4274, | | | |
| miR-4295 | | | |
| miR-203a-3p | PD-MCI and HC | * | * |
| hsa-miR-3173-3p, | MSA and HC | The individual | |
| hsa-miR-4292, | | rankings were | |
| hsa-miR-140-3p, | | sequentially 1, 2, 3, 3, | |
| hsa-miR-16-2-3p, | | 3, 3 | |
| hsa-miR-3937, | | (The same applied to | |
| hsa-miR-5093 | | the following) | |
| miR-4306, | MSA and PDND | 1, 2 | |
| miR-452-3p | | | |
| hsa-miR-758-5p | PDND and HC | | ** |
| hsa-miR-1197 | | | ** |
| hsa-miR-3173-3p, | MSA and HC | 1, 1, 3, 3, 5 | |
| hsa-miR-556-5p, | | | |
| hsa-miR-208b-5p, | | | |
| hsa-miR-5093, | | | |
| hsa-miR-4507 | | | |
| hsa-miR-4306, | PDND and MSA | 1, 2, 3, 3, 5, 5, 7, 7, 7, | |
| hsa-miR-452-3p, | | 7, 7 | |
| hsa-miR-648, | | | |
| hsa-miR-92b-5p, | | | |
| hsa-miR-3653-5p, | | | |
| hsa-miR-4782-3p, | | | |
| hsa-miR-302d-5p, | | | |
| hsa-miR-379-3p, | | | |
| hsa-miR-412-3p, | | | |
| hsa-miR-4296, | | | |
| hsa-miR-6747-3p | | | |
| hsa-miR-3667-3p, | PD and MSA + HC | 1, 4, 4, 4, 5 | |
| hsa-miR-3689a-5p, | | | |
| hsa-miR-3912-3p, | | | |
| hsa-miR-5187-3p, | | | |
| hsa-miR-548b-5p | | | |
| hsa-miR-519d-5p, | PD and HC | 1,2 | |
| hsa-miR-551b-3p | | | |

Grouping by extracellular vesicle proteins

| | | | |
|---|---|---|---|
| TAOK1 | Normal cognitive | *** | |
| | function (HC and | | |
| | PDND) vs. cognitive | | |
| | impairment (PDD and | | |
| | PD-MCI); | | |
| | Normal cognitive | *** | |
| | function (HC) vs. | | |
| | cognitive impairment | | |
| | (AD and MCI); | | |
| | Normal cognitive | | *** (p < 0.001) |
| | function (HC and | | |
| | PDND) vs. cognitive | | |
| | impairment (PDD and | | |
| | AD); | | |
| LCAT | MSA and HC | | |
| SERPINA4 | MSA and HC | | |
| CSEIL | MSA and HC | *** | |
| CRKL | MSA and HC | *** | |
| SERPINA4 | MSA and HC | 1 | * |
| | | | (P = 0.0127) |
| SERPINA4 | MSA and PD | | |
| ABCC4 | MSA and PD | ** | |
| ALDH4A1 | MSA and PD | *** | |
| APOE | MSA and PD | *** | |
| TINAGL1, CXCR1, | PD and HC | 1, 5,7,10 | |
| SWAP70, ADGRL2 | | | |
| Ykt6, CIDEB | PDND and PD-MCI + | 2, 1 | |
| | PDD | | |
| CIDEB, CD96, Ykt6, | PDND and PD-MCI | 1, 1, 2, 6 | |
| GLTPD2 | | | |
| CD69, SLC22A23, | PD-MCI and HC | 4, 4, 4, 4, 12 | |
| Tspan15, TTC7B, | | | |
| ST3GAL6 | | | |
| SAMD9, TTC7B, | AD+MCI and HC | 4, 4, 5, 11, 13 | |
| GNB1, ACTBL2, | | | |
| DOK3 | | | |
| eIF3B, SLC6A4, | PD and HC + MSA | 1, 1, 3, 1, 5, 1, 5, 5, 5, | |
| IQGAP1, TINAGLI, | | 4, 1, 7, 12, 12, 1, 4, 12, | |
| RPL18A, ABCC4, | | 12 | |
| CLCN5, MME, PUS1, | | | |
| ADIPOQ, MAP2K6, | | | |
| ACTR10, CBLN4, | | | |
| EPN1, LCAT, FUCA2, | | | |
| SNX8, CD3D | | | |
| eIF3B, TINAGLI, | PD and non-PD | 1, 1, 4, 4, 4, 7 | |
| ADIPOQ, FCGRT, | | | |
| FUCA2, ACTR10 | | | |
| LRRFIP2, ARL5A | AD + MCI and | 1,2 | |
| | PD-MCI + PDD | | |
| LRRFIP2, TINAGLI | AD + MCI and PDND | 1,1 | |
| CRKL, SLC6A4, | MSA and PDND | 1, 1, 4, 5, 5 | |
| ARF6, GNB1, | | | |
| ATP6V0D1 | | | |

In Table 5, AD meant Alzheimer's disease.
* (p < 0.05);
** (p < 0.01); and
*** (p < 0.001).

Please refer to Table 5 again. The aforementioned results showed that through the fitting verification of the prediction model and the 5-fold iterations of cross-validation of the prediction model, the optimized tuning parameters with the highest average AUC values were obtained. After the aforementioned optimized tuning parameters were obtained, then the BOLD selector was used for analyzing and identifying all factors with non-zero coefficients greater than or equal to the optimized tuning parameters on the delta axis to screen candidate biomarkers from the processed microRNA dataset or extracellular vesicle protein protein profile (as shown in the results of Table 5). The following was a detailed description of the individual screened biomarkers:

microRNA Biomarkers (Screening Phase)

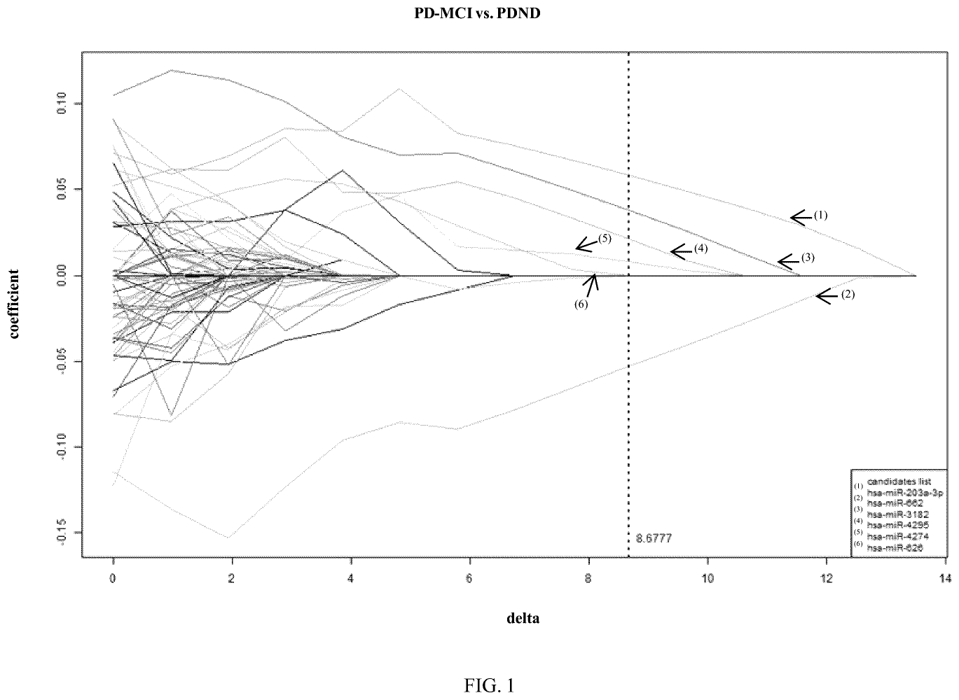

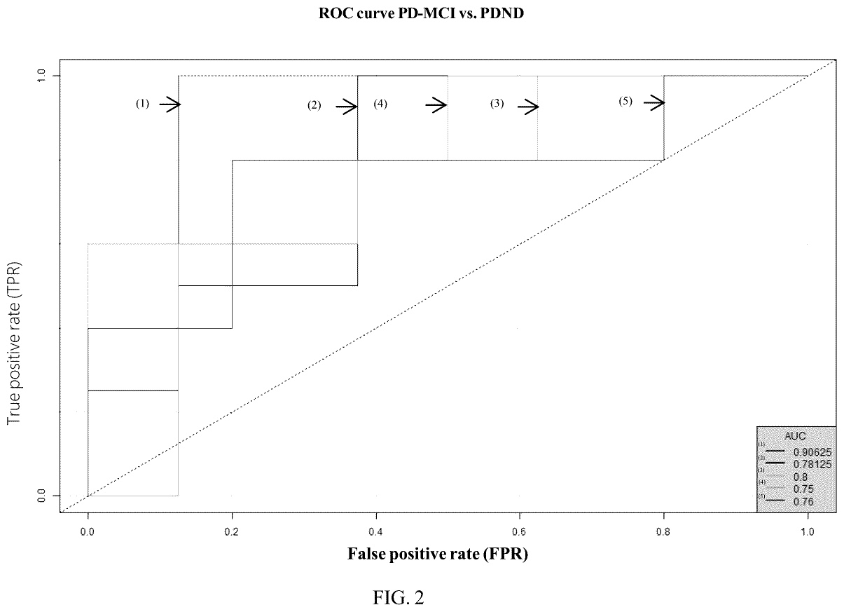

Please refer to Table 5 again, miR-203a-3p, miR-626, miR-662, miR-3182, miR-4274 and miR-4295 were screened to distinguish the PD-MCI cohorts from the PDND cohorts. Please refer to FIG. 1 and Table 5 together. FIG. 1 was a schematic diagram of the results of candidate microRNAs screened by the BOLD selector algorithm under the condition of an optimized tuning parameter of 8.6777 (y-axis represented a coefficient, and x-axis represented delta). Please refer to FIG. 2, it was a diagram showing the ROC analysis results obtained by the 5-fold iterations of cross-validation of the prediction model, which showed that the average AUC value was about 0.8.

Please refer to Table 5 again. In the screening phase, miR-203a-3p was screened to distinguish the PD-MCI cohorts and the HC cohorts (*p<0.05), wherein under 5-fold iterations of cross-validation of the prediction model, it was obtained that the average AUC value was about 0.8, and the screening results were obtained under the condition that the optimized tuning parameter with the highest average AUC value was 8.67.

Please refer to Table 5 again, hsa-miR-3173-3p, hsa-miR-4292, hsa-miR-140-3p, hsa-miR-16-2-3p, hsa-miR-3937 and hsa-miR-5093 were screened to distinguish the MSA cohorts from the HC cohorts, wherein the screening results were obtained under the condition that the optimized tuning parameter with the highest average AUC value was 11.341. The screened candidate microRNA was substituted into the logistic regression formula to calculate a prediction probability formula for disease grouping: f(x)=ln(p/(1−p)), p=e\hat{ }f(x)/(1+e\hat{ }f(x)), and specifically, an exemplary prediction probability formula for disease grouping: −0.84175+0.25292*(hsa-miR-3173-3p), wherein the aforementioned (hsa-miR-3173-3p) was represented by the content of the microRNA thereof in the sample.

Please refer to Table 5 again, miR-4306 and miR-452-3p were screened to distinguish MSA cohorts from PDND cohorts. The screening results were obtained under the condition that the optimized tuning parameter with the highest average AUC value was 10.1755.

Please refer to Table 5 again, hsa-miR-3173-3p, hsa-miR-556-5p, hsa-miR-208b-5p, hsa-miR-5093 and hsa-miR-4507 were screened to distinguish MSA cohorts from HC cohorts, wherein the screening results were obtained under the condition that the optimized tuning parameter with the highest average AUC value was 9.6236.

Please refer to Table 5 again, hsa-miR-4306, hsa-miR-452-3p, hsa-miR-648, hsa-miR-92b-5p, hsa-miR-3653-5p, hsa-miR-4782-3p, hsa-miR-302d-5p, hsa-miR-379-3p, hsa-miR-412-3p, hsa-miR-4296 and hsa-miR-6747-3p were screened to distinguish PDND cohorts from MSA cohorts, wherein the screening results were obtained under the condition that the optimized tuning parameter was 8.7533.

Please refer to Table 5 again, hsa-miR-3667-3p, hsa-miR-3689a-5p, hsa-miR-3912-3p, hsa-miR-5187-3p, and hsa-miR-548b-5p were screened to distinguish PD cohorts from MSA+HC cohorts, wherein the screening results were obtained under the condition that the optimized tuning parameter was 14.953.

Please refer to Table 5 again, hsa-miR-519d-5p and hsa-miR-551b-3p were screened to distinguish the PD cohorts from the HC cohorts, wherein the screening results were obtained under the condition that the optimized tuning parameter was 11.8573.

### Extracellular Vesicle Proteins (Screening Phase)

Please refer to Table 5 and the schematic diagram on the left side of FIG. 5 again. TAOK1 was screened to distinguish cohorts of cognitively normal (HC and PDND) and cohorts of cognitive impairment (PDD and PD-MCI) (*** p<0.001). Please refer to Table 5 and the schematic diagram on the right side of FIG. 5 again, TAOK1 was screened to distinguish cohorts of cognitively normal (HC) and cohorts of cognitive impairment (AD and MCI) (** p<0.01). Wherein, the screening results were obtained under the condition that the optimized tuning parameter was 1.7787.

Please refer to Table 5 again, LCAT, SERPINA4, CSEIL and CRKL were screened to distinguish MSA cohorts from HC cohorts (*** p<0.001), wherein the individual screening results were obtained under the condition that the optimized tuning parameter was 30.4.

Please refer to Table 5 again, SERPINA4 was screened to distinguish MSA cohorts from HC cohorts (with a p value of 0.0127) (*p<0.05).

Please refer to Table 5 again, SERPINA4, ABCC4, ALDH4A1 and APOE were screened to distinguish MSA cohorts from PD cohorts (*** p<0.001), wherein the individual screening results were obtained under the condition that the optimized tuning parameter was 49.5253.

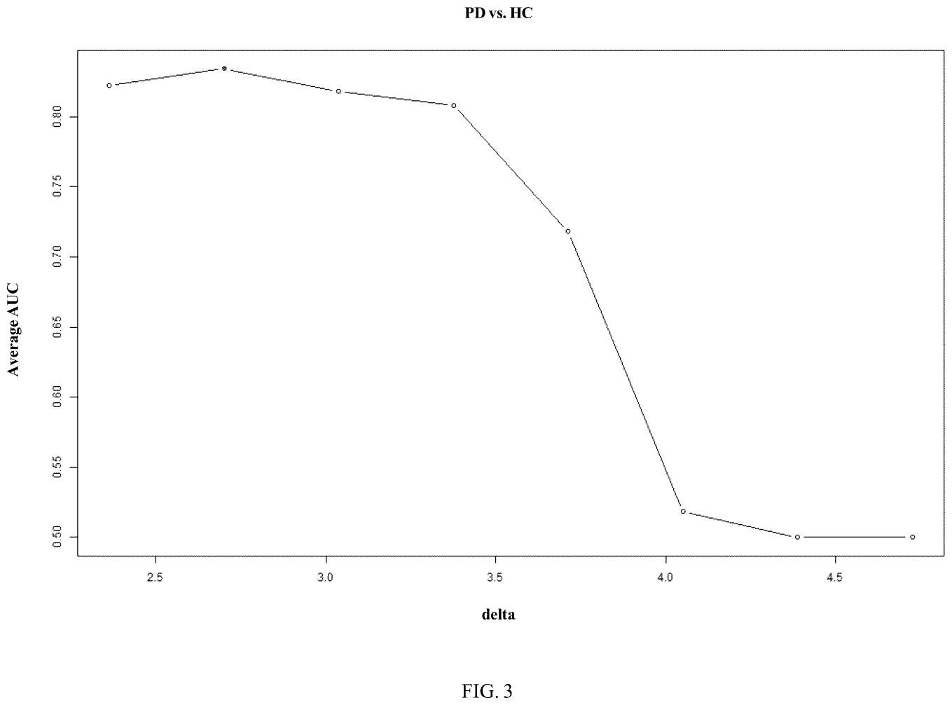

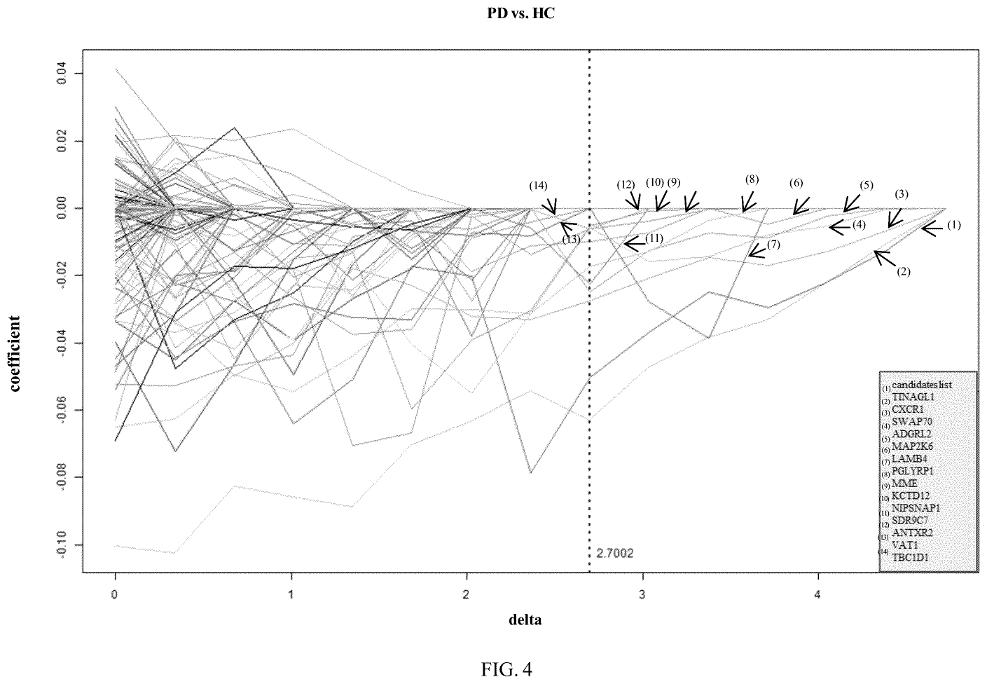

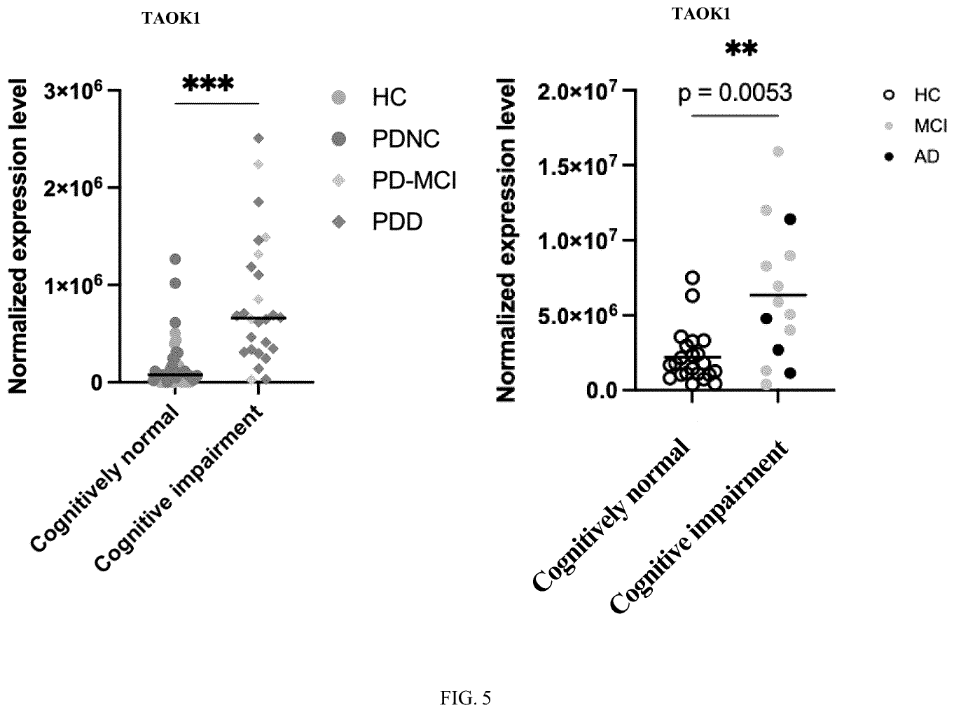

Please refer to Table 5 again, TINAGL1, CXCR1, SWAP70 and ADGRL2 were screened to distinguish PD cohorts from HC cohorts. Please refer to FIG. 3 together, it showed the average AUC value obtained under multiple iterations of cross-validation of the prediction model. The optimized tuning parameter was selected from a delta value corresponding to the highest average AUC (approximately 2.7 on the x-axis). Please refer to FIG. 4 together, it was a schematic diagram of the results of the candidate microRNAs screened by the BOLD selector algorithm under the condition that the optimized tuning parameter was 2.7002. The screened candidate extracellular vesicle protein was substituted into the logistic regression formula to calculate a prediction probability formula for disease grouping: f(x)=ln(p/(1−p)), p=e\hat{ }f(x)/(1+e\hat{ }f(x)), and specifically, an exemplary prediction probability formula for disease grouping: 1.653*1+−1.414*(0.308*(TINAGL1−267468.38/183983.58)+0.283*(CXCR1−657481.16/632718.85)+0.302*(SWAP70−216480.35/204242.15)+0.301*(ADGRL2−116523.76/98490.30)); wherein each extracellular vesicle protein in the formula was expressed by the protein content thereof in the sample.

Please refer to Table 5 again, Ykt6 and CIDEB were screened to distinguish the PDND cohorts from the PD-MCI+PDD cohorts, wherein the screening results were obtained under the condition that the optimized tuning parameter was 9.7494.

Please refer to Table 5 again, CIDEB, CD96, Ykt6 and GLTPD2 were screened to distinguish the PDND cohorts from the PD-MCI cohorts, wherein the screening results were obtained under the condition that the optimized tuning parameter was 7.8198.

Please refer to Table 5 again, CD69, SLC22A23, Tspan15, TTC7B and ST3GAL6 were screened to distinguish PD-MCI cohorts from HC cohorts, wherein the screening results were obtained under the condition that the optimized tuning parameter was 4.1577.

Please refer to Table 5 again, SAMD9, TTC7B, GNB1, ACTBL2 and DOK3 were screened to distinguish AD+MCI cohorts from HC cohorts, wherein the screening results were obtained under the condition that the optimized tuning parameter was 5.4654.

Please refer to Table 5 again, eIF3B, SLC6A4, IQGAP1, TINAGL1, RPL18A, ABCC4, CLCN5, MME, PUS1, ADIPOQ, MAP2K6, ACTR10, CBLN4, EPN1, LCAT, FUCA2, SNX8 and CD3D were screened to distinguish PD cohorts from HC+MSA cohorts, wherein the screening results were obtained under the condition that the optimized tuning parameter was 15.5125.

Please refer to Table 5 again, EIF3B, TINAGL1, ADIPOQ, FCGRT, FUCA2, and ACTR10 were screened to distinguish PD cohorts from non-PD cohorts, wherein the screening results were obtained under the condition that the optimized tuning parameter was 18.667.

Please refer to Table 5 again, LRRFIP2 and ARL5A were screened to distinguish AD+MCI cohorts from PD-MCI+PDD cohorts, wherein the screening results were obtained under the condition that the optimized tuning parameter was 11.4457.

Please refer to Table 5 again, LRRFIP2 and TINAGL1 were screened to distinguish AD+MCI cohorts from PDND cohorts, wherein the screening results were obtained under the condition that the optimized tuning parameter was 9.3772.

Please refer to Table 5 again, CRKL, SLC6A4, ARF6, GNB1 and ATP6V0D1 were screened to distinguish MSA cohorts from PDND cohorts, wherein the screening results were obtained under the condition that the optimized tuning parameter was 14.7124.

The data of Cohort 2 was divided into 5 parts for cross-validation, wherein 80% of the data was used for training of the prediction model, and the remaining data was used for detection of the prediction model.

Through the fitting verification of the prediction model and multiple iterations of cross-validation on the prediction model, the optimized tuning parameters with the highest average AUC values were obtained, and the optimized tuning parameters were used for re-screening of biomarkers to retain important and candidate biomarkers to calculate a final logistic regression formula.

### Example 7. Grouping of Participants According to Candidate Biomarkers (microRNA and Extracellular Vesicle Proteins)

In order to verify the grouping effect of the previously screened candidate biomarkers (as the target biomarkers to be tested in subsequent experiments) on the participants, the following test was conducted. By collecting plasma samples from the participants and detecting the expression level of the target biomarker, it was compared that whether the expression level of the target biomarker showed a statistically significant difference between the two cohorts.

The Part of Testing microRNAs
1. Extraction of RNAs from Participants

Plasma was collected as described in Example 3 above. Next, small RNAs were extracted from the plasma of the participants by using a miRNeasy reagent kit (Qiagen, Germany). The extraction of RNAs was carried out according to the usage process of the reagent kit with some modifications to the process as follows: the thawed plasma sample was subjected to a series of centrifugation steps: first, centrifugation at a rotation speed of 12,000×g at 4° C. for 3 minutes (at a fixed angle, KUBOTA 6200, Japan), and then further centrifugation at a rotation speed of 12,000×g (at a fixed angle, KUBOTA 3300T, Japan) at room temperature for 30 seconds, 30 seconds, 30 seconds, 2 minutes and 5 minutes. Next, a mini elution column (UCP MiniElute column, Qiagen, Germany) was used for isolating and purifying RNAs, wherein RNase-free water (Invitrogen, Thermo Fisher) preheated at 55° C. was used for column elution of RNAs. The eluted RNA was purified again with a mini elution column and incubated at room temperature for 10 minutes. Next, the RNA was centrifuged at a rotation speed of 12,000×g for 1 minute (at a fixed angle, KUBOTA 3300T, Japan), and then the final RNA was placed on ice for a subsequent reverse transcription (RT) reaction.

2. Synthesis of cDNA

A miRCURY LNA miRNA SYBR Green kit (Qiagen, Germany) was used as a reagent kit for the reaction. The synthesis of cDNA was carried out according to the usage process of the reagent kit. The synthesized cDNA samples were stored at −20° C. for ddPCR detection.

3. Use of Droplet Digital PCR (ddPCR) (Bio-Rad, USA) for nucleic acid amplification and detection. The ratio of the target miRNA was obtained by dividing the content of the target miRNA by the endogenous miRNA (e.g., miR-16-5p) content and then multiplying by 10,000.

### 4. Results

Please refer to Table 5 again, in the validation phase “Comparing the statistical significance (p value) of biomarker expression between two cohorts” in the rightmost column of Table 5, when the screened candidate biomarker miR-203a-3p was used as the target biomarker to be tested by ddPCR, the results showed that the expression level of miR-203a-3p showed a statistically significant difference between the PD-MCI cohort and the HC cohort (*p<0.05), indicating that the candidate miR-203a-3p could indeed be used as a biomarker to distinguish the PD-MCI cohort from the HC cohort.

Please refer to Table 5 again, when the screened candidate biomarkers hsa-miR-758-5p and hsa-miR-1197 were used as the target biomarkers to be tested by ddPCR, the results showed that the expression level of hsa-miR-758-5p and hsa-miR-1197 showed statistically significant differences between the PDND cohort and the HC cohort, respectively (** p<0.01), indicating that the candidate hsa-miR-758-5p and hsa-miR-1197 could indeed be used as biomarkers to distinguish the PDND cohort from the HC cohort.

### Determination of Extracellular Vesicle Proteins

1. Purification of extracellular vesicle proteins, basically referring to the aforementioned Example 5. An enzyme-linked immunosorbent assay (ELISA) was utilized to analyze whether the target extracellular vesicle protein was expressed in the sample and to analyze the expression level of the target extracellular vesicle protein. The model of the ELISA kit for testing TAOK1 was (OKEH03485, Aviva System Biology), and the other ELISA kits for detecting extracellular vesicle proteins were all available in the market. The experimental procedure mainly referred to the instruction manual attached to the ELISA kit.

### 2. Results

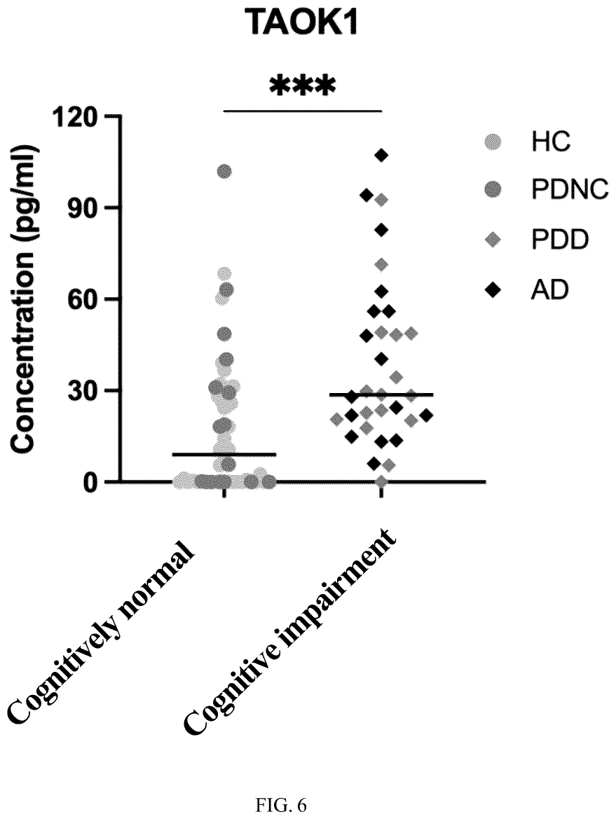

Please refer to FIG. 6 and the validation phase “Comparing the statistical significance (p value) of biomarker expression between two cohorts” in the rightmost column of Table 5, when the screened candidate biomarker TAOK1 was used as the target biomarker to be tested by ELISA, the results showed that the expression level of TAOK1 showed a statistically significant difference (*** p<0.001) between the cohort with cognitively normal (HC and PDND) and the cohort with cognitive impairment (PDD and AD), indicating that the candidate TAOK1 could indeed be used as a biomarker to distinguish the aforementioned cohort with cognitively normal from the aforementioned cohort with cognitive impairment.

Please refer to Table 5 again. When the screened candidate biomarkers LCAT, SERPINA4, CSEIL and CRKL were respectively used as the target biomarkers to be tested by ELISA, the results showed that the expression level of LCAT, SERPINA4, CSEIL and CRKL showed statistically significant differences (*** p<0.001) between the MSA cohort and the HC cohort, indicating that the candidate LCAT, SERPINA4, CSEIL and CRKL could indeed be used as biomarkers to distinguish the MSA cohort from the HC cohort.

Please refer to Table 5 again. When the screened candidate biomarker SERPINA4 was used as the target biomarker to be tested by ELISA, the results showed that the expression level of SERPINA4 showed a statistically significant difference (*p<0.05) between the MSA cohort and the HC cohort, indicating that the candidate SERPINA4 could indeed be used as a biomarker to distinguish the MSA cohort from the HC cohort.

Please refer to Table 5 again. When the screened candidate biomarkers SERPINA4, ABCC4, ALDH4A1 and ApoE were respectively used as the target biomarkers to be tested by ELISA, the results showed that the individual expression level of SERPINA4, ABCC4, ALDH4A1 and ApoE showed statistically significant differences (*** p<0.001) between the MSA cohort and the PD cohort, indicating that the candidate SERPINA4, ABCC4, ALDH4A1 and APOE could indeed be used as biomarkers to distinguish the MSA cohort from the PD cohort.

In view of the above, the method for screening a biomarker for differential diagnosis of the status of Parkinson's disease and/or Parkinsonism and the computer system for executing the aforementioned method as mentioned in the present invention can correctly diagnose and predict the status of an individual suffering from Parkinson's disease when the dataset is relatively small and there are many potential influencing factors. It can also be implemented in many biomarker identification processes based on other clinical samples. Besides, the aforementioned method has a basis for evaluating whether biomarkers such as microRNAs and EV proteins can effectively distinguish subtypes of Parkinson's disease (for example: the results predicted by the prediction model are compared with the patient grouping results under clinical detection data), and the biomarkers screened by the aforementioned method can be used for differential diagnosis of patients with Parkinsonism and group them, which is beneficial to the early diagnosis and precise treatment of the patients.

The present disclosure has been described in detail above. However, what is described above is only some of the preferred embodiments of the present disclosure and should not be considered to limit the scope of implementation of the present disclosure. That is, all equivalent changes and modifications made according to the claims of the present disclosure should still fall within the scope of the patent coverage of the present disclosure.

What is claimed is:

1. A method for screening a biomarker or biomarkers for differential diagnosis of the status of Parkinson's Disease (PD), Parkinson's disease with mild cognitive impairment, Parkinson's disease dementia, Alzheimer's disease, and/or multiple system atrophy, comprising:
a) acquiring plasma samples of a plurality of individuals to obtain a plurality of relevant data of these individuals;
b) isolating ribonucleic acids containing micro ribonucleic acids (microRNAs) and extracellular vesicular proteins (EV proteins) from the plasma samples of the individuals, and identifying and quantifying microRNAs and EV proteins to obtain respective profiles;
c) using a Biomedical Oriented Logistic Dantzig Selector (BOLD Selector) to screen candidate microRNA(s) from the microRNA profile, and to screen candidate EV protein(s) from the EV protein profile; and
d) calculating a logistic regression formula according to the candidate microRNA and the candidate extracellular vesicle protein to establish a prediction model, and using the prediction model to predict the status of Parkinson's disease, Parkinson's disease with mild cognitive impairment, Parkinson's disease dementia, Alzheimer's disease, and/or multiple system atrophy in these individuals.

2. The method of claim 1, wherein in the step a), the types of grouping of these individuals comprise: Parkinson's Disease patients with normal cognition ability (no Dementia) (PDND), PD patients with mild cognitive impairment (PD-MCI), Parkinson's Disease Dementia (PDD), Multiple system atrophy (MSA), Alzheimer's disease (AD), and healthy individuals (HC).

3. The method of claim 1, wherein the relevant data is selected from a group consisting of: Movement disorder society-Unified Parkinson's disease rating scale (MDS-UPDRS), Montreal Cognitive Assessment (MoCA) and Mini-mental status examination (MMSE), Unified Multiple System Atrophy Rating Scale (UMSARS), physical data and medical history data.

4. The method of claim 3, wherein the physical data comprises age, gender, education level, living habits, diet and exercise habits, and the medical history data comprises medication records, age of onset of Parkinson's disease, and disease duration of Parkinson's disease.

5. The method of claim 1, wherein the microRNA is selected from a group consisting of: miR-203a-3p, miR-626, miR-662, miR-3182, miR-4274, miR-4295, hsa-miR-3173-3p, miR-4306, miR-452-3p, hsa-miR-758-5p, hsa-miR-1197, hsa-miR-208b-5p, hsa-miR-4507, hsa-miR-648, hsa-miR-92b-5p, hsa-miR-3667-3p, hsa-miR-3689a-5p, hsa-miR-3912-3p, hsa-miR-5187-3p, hsa-miR-548b-5p, hsa-miR-519d-5P and hsa-miR-551b-3p.

6. The method of claim 1, wherein the extracellular vesicle protein is selected from a group consisting of: TAOK1 (Serine/threonine-protein kinase TAO1), LCAT (Lecithin cholesterol acyl transferase), CSEIL (Cellular Apoptosis Susceptibility protein, also known as CAS), CRKL (CRK-like proto-oncogene, an adaptor protein), SERPINA4 (Serpin Family A Member 4, also known as Kallistatin), APOE (Apolipoprotein E), ABCC4 (ATP-binding cassette subfamily C member 4), ALDH4A1 (aldehyde dehydrogenase 4 family member A1), TINAGL1 (Tubulointerstitial Nephritis Antigen Like 1), CXCR1 (a chemokine (C-X-C motif) receptor), SWAP70 (Switching B Cell Complex Subunit, 70 kDa), ADGRL2 (Adhesion G Protein-Coupled Receptor L2), Synaptobrevin homolog YKT6, CIDEB (Cell death-inducing DFFA-like effector B), CD96, GLTPD2, CD69, SLC22A23, Tspan15 (transmembrane protein 15), TTC7B, ST3GAL6 (ST3 Beta-Galactoside Alpha-2,3-Sialyltransferase 6), SAMD9, TTC7B, GNB1, ACTBL2 (actin beta like 2), DOK3 (docking protein 3), eIF3B (eukaryotic initiation factor 3), IQGAP1 (IQ domain GTPase-activating protein 1), RPL18A (human 60S ribosomal protein L18a), CLCN5 (Chloride Channel Protein 5), MME (membrane metalloendopeptidase), PUS1, ADIPOQ (Adiponectin), MAP2K6 (Dual Specificity Mitogen-activated Protein Kinase 6), CBLN4 (ACTR10, Cerebellin 4), Epsin 1 (endocytosis accessory protein 1, EPN1), FUCA2 (Alpha-L-fucosidase 2), SNX8, CD3D (CD3 δ subunit of T cell receptor complex), FCGRT, LRRFIP2 (LRR binding FLII interacting protein 2), ARFLP5 (ADP-ribosylation Factor-like Protein 5A), SLC6A4, ARF6 (Switch II GTPase protein) and ATP6V0D1 (ATPase H+ transporting V0 subunit d1).

7. The method of claim 1, wherein before performing the step c), the method further comprises: conducting a data pre-processing step to obtain a processed dataset for the Biomedical Oriented Logistic Dantzig Selector; wherein, when at least one data is missing from the processed dataset, a minimum reading value in other data is inspected and selected in a sample corresponding to the missing data, and an interval between the minimum reading value and zero is uniformly cut to obtain an imputed value, which is then used for filling up the missing data according to the overall averages of candidates without missing values.

8. The method of claim 1, wherein in the step c), the method further comprises: providing an optimized tuning parameter, and then using the Biomedical Oriented Logistic Dantzig Selector to analyze and identify all factors with non-zero coefficients and the shrink-to-zero position being greater than or equal to the optimized tuning parameter on a delta axis, so as to screen the candidate microRNA from the processed microRNA dataset, and screen the candidate extracellular vesicle protein from the extracellular vesicle protein profile.

9. The method of claim 1, wherein in the step d), the Parkinson's disease and/or Parkinsonism is selected from a group consisting of: Parkinson's Disease patients with normal cognition ability (no Dementia) (PDND), PD patients with mild cognitive impairment (PD-MCI), Parkinson's Disease Dementia (PDD), Multiple system atrophy (MSA), Alzheimer's disease (AD), and healthy individuals (HC).

10. The method of claim 1, wherein in the step d), the logical regression formula adopts a combination of weighted value of a set of microRNAs, or a combination of weighted value of a set of extracellular vesicle proteins.

11. The method of claim 1, further comprising, after the step d), a step of conducting 5-fold iterations of cross-validation on the prediction model.

12. The method of claim 11, wherein the cross-validation step comprises training the prediction model to evaluate the predictive ability of the prediction model for the status of Parkinson's disease, Parkinson's disease with or without cognitive impairment and/or Parkinson's disease dementia compared to the grouping results of the individuals in the step a).

13. The method of claim 11, wherein the cross-validation step comprises a detection of the prediction model, wherein the statistical indicators of the detection comprises: sensitivity, specificity, accuracy and area under ROC curve (AUC).

14. The method of claim 1, wherein the method is implemented by a computer.

15. A data analytic scheme for executing the method of claim 1.

16. A biomarker for differential diagnosis of the status of Parkinson's disease, Parkinson's disease with mild cognitive impairment and/or Parkinson's disease dementia, wherein the biomarker is a microRNA and/or an extracellular vesicle protein.

17. The biomarker of claim 16, wherein the microRNA is selected from a group consisting of: miR-203a-3p, miR-626, miR-662, miR-3182, miR-4274, miR-4295, hsa-miR-3173-3p, miR-4306, miR-452-3p, hsa-miR-758-5p, hsa-miR-1197, hsa-miR-208b-5p, hsa-miR-4507, hsa-miR-648, hsa-miR-92b-5p, hsa-miR-3667-3p, hsa-miR-3689a-5p, hsa-miR-3912-3p, hsa-miR-5187-3p, hsa-miR-548b-5p, hsa-miR-519d-5P and hsa-miR-551b-3p.

18. The biomarker of claim 16, wherein the extracellular vesicle protein is selected from a group consisting of: TAOK1 (Serine/threonine-protein kinase TAO1), LCAT (Lecithin cholesterol acyl transferase), CSEIL (Cellular Apoptosis Susceptibility protein, also known as CAS), CRKL (CRK-like proto-oncogene, an adaptor protein), SERPINA4 (Serpin Family A Member 4, also known as Kallistatin), APOE (Apolipoprotein E), ABCC4 (ATP-binding cassette subfamily C member 4), ALDH4A1 (aldehyde dehydrogenase 4 family member A1), TINAGL1 (Tubulointerstitial Nephritis Antigen Like 1), CXCR1 (a chemokine (C-X-C motif) receptor), SWAP70 (Switching B Cell Complex Subunit, 70 kDa), ADGRL2 (Adhesion G Protein-Coupled Receptor L2), Synaptobrevin homolog YKT6, CIDEB (Cell death-inducing DFFA-like effector B), CD96, GLTPD2, CD69, SLC22A23, Tspan15 (transmembrane protein 15), TTC7B, ST3GAL6 (ST3 Beta-Galactoside Alpha-2,3-Sialyltransferase 6), SAMD9, TTC7B, GNB1, ACTBL2 (actin beta like 2), DOK3 (docking protein 3), eIF3B (eukaryotic initiation factor 3), IQGAP1 (IQ domain GTPase-activating protein 1), RPL18A (human 60S ribosomal protein L18a), CLCN5 (Chloride Channel Protein 5), MME (membrane metalloendopeptidase), PUS1, ADIPOQ (Adiponectin), MAP2K6 (Dual Specificity Mitogen-activated Protein Kinase 6), ACTR10, CBLN4 (Cerebellin 4), Epsin 1 (endocytosis accessory protein 1, also known as EPN1), FUCA2 (Alpha-L-fucosidase 2), SNX8, CD3D (CD3 δ subunit of T cell receptor complex), FCGRT, LRRFIP2 (LRR binding FLII interacting protein 2), ARFLP5 (ADP-ribosylation Factor-like Protein 5A), SLC6A4, ARF6 (Switch II GTPase protein) and ATP6V0D1 (ATPase H+ transporting V0 subunit d1).
